(19) United States
(12) Patent Application Publication
(10) Pub. No.: US 2009/0131753 A1
(43) Pub. Date: May 21, 2009

(54) PULLING APPARATUS AND ENDOSCOPE SYSTEM

(76) Inventors: Shoichi AMANO, Hachioji-shi (JP); Seigo ITO, Hachioji-shi (JP); Atsushi OGAWA, Hachioji-shi (JP); Takashi SAWAI, Hachioji-shi (JP); Harutaka ADACHI, Ome-shi (JP); Masanobu KOITABASHI, Hachioji-shi (JP)

Correspondence Address: OSTROLENK FABER GERB & SOFFEN, 1180 AVENUE OF THE AMERICAS, NEW YORK, NY, 100368403, US

(21) Appl. No.: 12/269,405

(22) Filed: Nov. 12, 2008

(30) Foreign Application Priority Data
Nov. 15, 2007 (JP) 2007-296994

(51) Int. Cl. A61B 1/01; B65H 77/00

(52) U.S. Cl. 600/118; 254/387

(57) ABSTRACT

A pulling apparatus includes an input portion configured to be operated, a braking portion configured to apply a braking force to the input portion to generate a resistance force against to an operation to the input portion, a pulling portion configured to pull a pulling member, a pulling order generation portion configured to generate a control order signal to the pulling portion such that the pulling member is pulled by the pulling portion in accordance with an operation to the input portion, a pulling amount detecting portion configured to detect pulling amount of the pulling member, a pulling amount increase and decrease determination portion configured to determine an increase and decrease in the pulling amount detected by the pulling amount detecting portion, and a braking control portion configured to control the braking portion in accordance with a determination result obtained by the pulling amount increase and decrease determination portion.

### CROSS-REFERENCE TO RELATED APPLICATIONS

This application is based upon and claims the benefit of priority from prior Japanese Patent Application No. 2007-296994, filed Nov. 15, 2007, the entire contents of which are incorporated herein by reference.

### BACKGROUND OF THE INVENTION

1. Field of the Invention

The present invention relates to a pulling apparatus configured to pull a pulling member in accordance with an operation to an input portion and an endoscope system including the pulling apparatus.

2. Description of the Related Art

Various kinds of pulling apparatuses has been used, which is configured to pull a pulling member in accordance with an operation to an input portion.

Jpn. Pat. Appln. KOKAI Publication No. 2003-275168 discloses an electric bending endoscope apparatus having a pulling apparatus. In an endoscope of the electric bending endoscope apparatus, an operation portion configured to be held and operated by an operator is coupled with a proximal end portion of an elongated insertion portion configured to be inserted into the interior of the body. A bending portion configured to be actuated to bend is arranged at a distal end portion of the insertion portion. Angle wires extended from the bending portion are inserted through the insertion portion to be led into the operation portion and wound around sprockets in the operation portion. The sprockets are driven by an electric motor. When operating a track ball arranged in the operation portion, the electric motor is driven, the sprockets are rotated, the angle wires are pulled and loosened, and so the bending portion is actuated to bend. Here, marker portions configured to be detected by an optical sensor are arranged on the angle wires, and it is possible to detect whether the bending portion is not being bent based on whether the angle wires are not pulled. Further, an electromagnetic brake is arranged in the operation portion and is configured to apply a braking force to the track ball to generate a resistance force against an operation to the track ball. An electromagnetic brake normally applies a fixed braking force to the track ball, but releases braking when it is detected that the bending portion is not being bent. Therefore, it is possible to recognize whether the bending portion is not being bent based on a change in resistance force against an operation to the track ball.

### BRIEF SUMMARY OF THE INVENTION

In an aspect of the present invention, a pulling apparatus includes: an input portion configured to be operated; a braking portion configured to apply a braking force to the input portion to generate a resistance force against to an operation to the input portion; a pulling portion configured to pull a pulling member; a pulling order generation portion configured to generate a control order signal to the pulling portion such that the pulling member is pulled by the pulling portion in accordance with an operation to the input portion; a pulling amount detecting portion configured to detect pulling amount of the pulling member; a pulling amount increase and decrease determination portion configured to determine an increase and decrease in the pulling amount detected by the pulling amount detecting portion; and a braking control portion configured to control the braking portion in accordance with a determination result obtained by the pulling amount increase and decrease determination portion.

In another aspect of the present invention, an endoscope system includes: an input portion configured to be operated; a braking portion configured to apply a braking force to the input portion to generate a resistance force against an operation to the input portion; a pulling member; a pulling portion configured to pull the pulling member; a pulling order generation portion configured to generate a control order signal to the pulling portion such that the pulling member is pulled by the pulling portion in accordance with an operation to the input portion; a bending portion configured to be actuated to bend by the pulling member in accordance with pulling of the pulling member by the pulling portion; a pulling amount detecting portion configured to detect pulling amount of the pulling member; a pulling amount increase and decrease determination portion configured to determine an increase and decrease in the pulling amount detected by the pulling amount detecting portion; and a braking control portion configured to control the braking portion in accordance with a determination result obtained by the pulling amount increase and decrease determination portion.

### BRIEF DESCRIPTION OF THE SEVERAL VIEWS OF THE DRAWING

The accompanying drawings, which are incorporated in and constitute a part of the specification, illustrate embodiments of the invention, and together with the general description given above and the detailed description of the embodiments given below, serve to explain the principles of the invention.

FIG. 1 is a perspective view showing an endoscope system according to an embodiment of the present invention;

FIG. 2 is a perspective view showing an operation portion according to the embodiment of the present invention;

FIG. 3 is a front view showing a track ball according to the embodiment of the present invention;

FIG. 4A is a side view showing a braking portion according to the embodiment of the present invention;

FIG. 4B is a cross-sectional view showing the braking portion according to the embodiment of the present invention taken along a line IVB-IVB in FIG. 4A;

FIG. 4C is a cross-sectional view showing the braking portion according to the embodiment of the present invention taken along a line IVC-IVC in FIG. 4A;

FIG. 4D is a front view showing the braking portion according to the embodiment of the present invention;

FIG. 5 is a block diagram showing an endoscope system according to the embodiment of the present invention;

FIG. 6 is a graph showing a propulsive amount characteristic according to the embodiment of the present invention;

FIG. 7 is a circuit diagram showing a driving motor according to the embodiment of the present invention;

FIG. 8A is a timing chart showing a driving pulse in a single phase excitation mode of the driving motor according to the embodiment of the present invention;

FIG. 8B is a timing chart showing a driving pulse in a two phase excitation mode of the driving motor according to the embodiment of the present invention; and

FIG. 8C is a timing chart showing a driving pulse in a single-two phase excitation mode of the driving motor according to the embodiment of the present invention.

### DETAILED DESCRIPTION OF THE INVENTION

An embodiment according to the present invention will now be explained hereinafter with reference to the accompanying drawings.

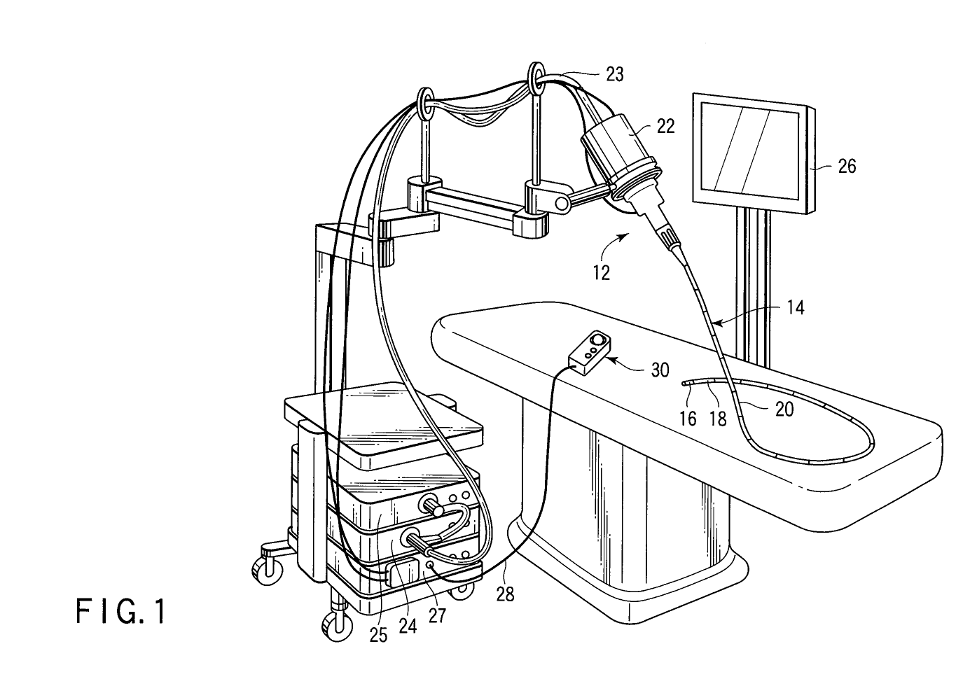

FIGS. 1 to 8C show an embodiment according to the present invention.

Referring to FIG. 1, an endoscope 12 in an endoscope system includes an elongated insertion portion 14 configured to be inserted into the interior of the body. In the insertion portion 14, a distal end rigid portion 16 having rigidity, a bending portion 18 configured to be actuated to bend in upper, lower, left, and right directions, and a insertion tube portion 20 long and having flexibility are sequentially provided from a distal end side to a proximal end side. A proximal end portion of the insertion portion 14 is detachably coupled with a driving unit 22. Angle wires configured to actuate the bending portion 18 to bend are inserted through the insertion portion 14 from the bending portion 18 to the proximal end portion of an insertion portion 14. When the angle wires are pulled and loosened by the driving unit 22, the bending portion 18 is actuated to bend. A universal cord 23 is extended from the driving unit 22, and the universal cord 23 is connected with a light source device 24 and a video processor 25. Illumination light is generated by the light source device 24, and is transmitted through a light guide inserted through the endoscope 12, and is applied to an observation target from a distal end portion of the endoscope 12. An observation image is picked up by an image pick-up unit at the distal end portion of the endoscope 12, an image signal is transmitted from the image pick-up unit through a signal line inserted through the endoscope 12 to output to the video processor 25, and the video processor 25 processes the image signal to display an observation image in a monitor 26. Further, a system controller 27 is connected with the video processor 25. An operation portion 30 is connected with the system controller 27 through an operation cord 28.

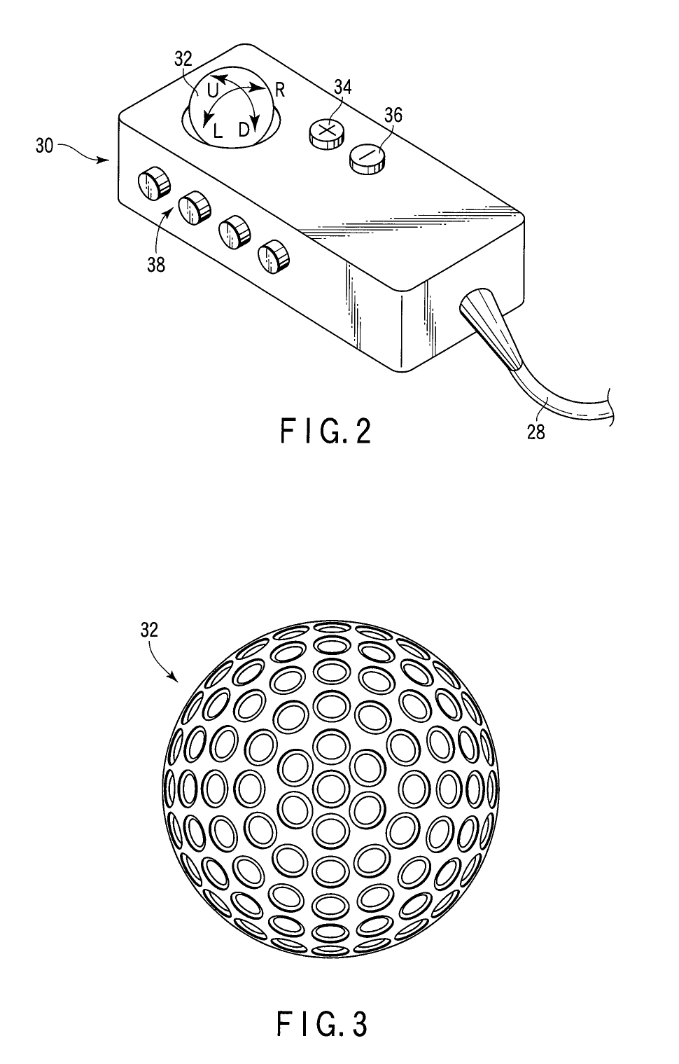

The operation portion 30 will be explained with reference to FIG. 2. A track ball 32 is arranged in the operation portion 30 and an input for actuating the bending portion 18 to bend is to be performed by the track ball 32 as an input portion. It is to be noted that a return-type joystick may be used in place of the track ball 32. The track ball 32 is rotatable in arbitrary directions. The track ball 32 includes an up/down direction and a left/right direction perpendicular to each other and indicated by arrows UD and LR in the drawing. The bending portion 18 is actuated to bend in the up/down direction and the left/right direction with a bending amount corresponding with an up/down direction component and a left/right direction component in a rotating operation amount of the track ball 32. Furthermore, a conversion ratio increasing switch 34 and a conversion ratio decreasing switch 36 are arranged in the operation portion 30 and are configured to adjust a conversion ratio of a bending amount of the bending portion 18 with respect to a rotating operation amount to the track ball 32. Further, various kinds of switches 38 configured to operate, e.g., an image pick-up actuation of the endoscope 12 are arranged in the operation portion 30.

The track ball 32 will now be explained with reference to FIG. 3. A friction increasing portion is formed on an entire outer surface of the track ball 32 and is configured to increase friction to avoid slippage at the time of a rotating operation. In this embodiment, dimpling is performed on the entire outer surface of the track ball 32. Alternatively, blasting may be performed.

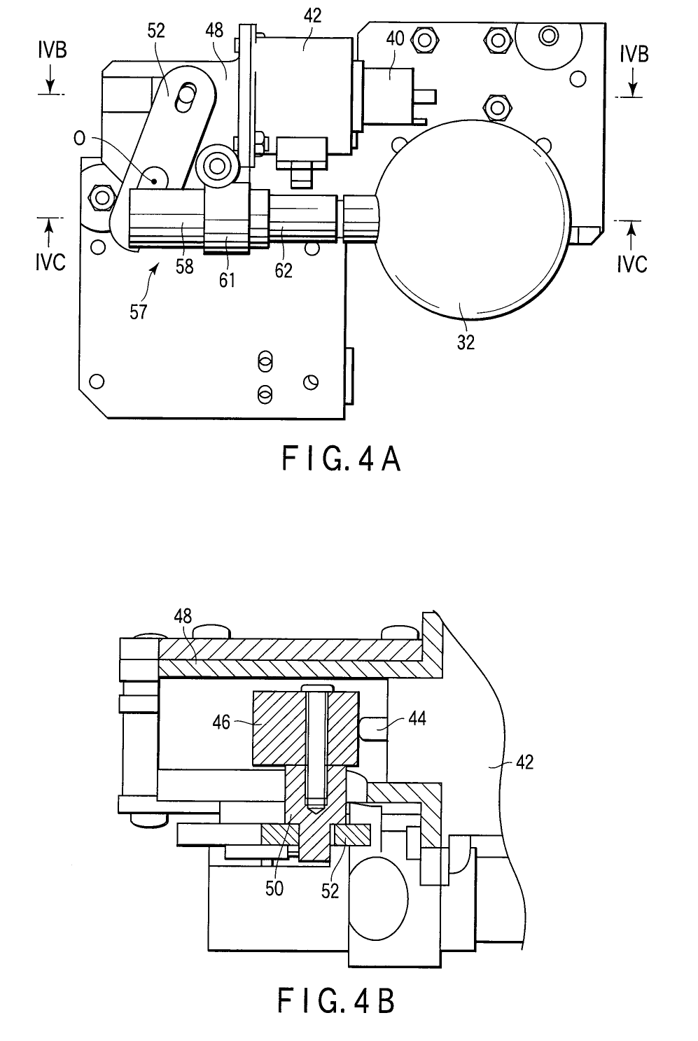

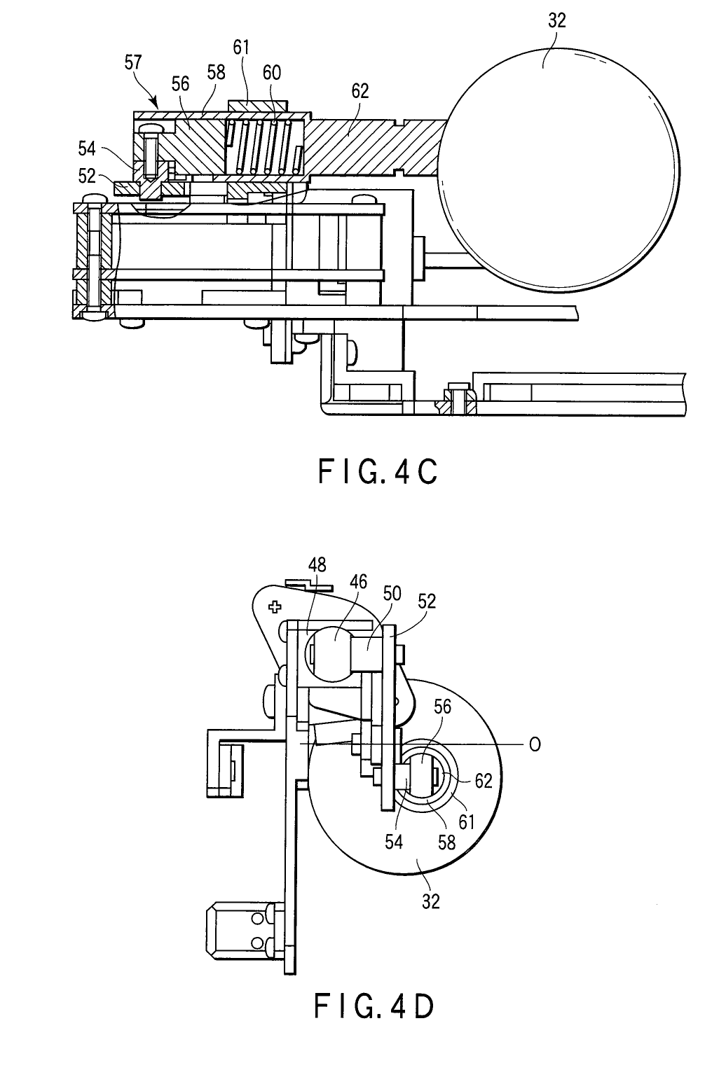

A braking portion configured to generate a resistance force with respect to a rotating operation to the track ball 32 will now be explained with reference to FIGS. 4A to 4D.

The braking portion includes a linear actuator 42 of a stepping motor scheme. That is, the linear actuator 42 is configured to convert a rotating motion of a braking motor 40 as a stepping motor into a propulsive motion. The linear actuator 42 is coupled with a braking member 57 configured to apply a braking force to the track ball 32, through a link mechanism configured to increase a propulsive force. That is, an output shaft 44 of the linear actuator 42 is coupled with a first piston 46. The first piston 46 is movable forward and backward in a propulsive direction of the output shaft 44 in a first cylinder 48. A first end portion of a link 52 is coupled with the first piston 46 through a first coupling member 50, and a second piston 56 is coupled with a second end portion of the link 52 through a second coupling member 54. Here, the link 52 is rotatable around a central axis O of the link 52, and a distance between the central axis O of the link 52 and the second end portion is smaller than a distance between the central axis O thereof and the first end portion. Furthermore, the second piston 56 is accommodated movably forward and backward in an axial direction of the braking member 57 in a second cylinder 58 on one end side of the braking member 57 and is connected with a brake portion 62 on the other end side of the braking member 57 through a compression spring 60 in the second cylinder 58. The braking member 57 is extended from a position near the second end portion of the link 52 in a direction parallel and opposite to the propulsive direction of the output axis 44 of the linear actuator 42, and is supported by a support portion 61 movably forward and backward in an axial direction thereof. The brake portion 62 of the braking member 57 is in contact with the track ball 32.

By a propulsive motion of the output shaft 44 of the linear actuator 42, the first piston 46 is propelled, the link 52 is rotated, and the second piston 56 is propelled. Here, based on the principle of leverage of the link 52, the propulsive force is increased and transmitted from the first piston 46 to the second piston 56. The compression spring 60 is compressed by the second piston 56 in accordance with a propulsive amount of the second piston 56, a spring force corresponding with a compression amount is applied to the brake portion 62 of the braking member 57 from the compression spring 60, and a braking force corresponding with the spring force is applied to the track ball 32 from the brake portion 62. Furthermore, a resistance force corresponding with the braking force is generated against a rotating operation to the track ball 32. In this manner, the resistance force against the rotating operation to the track ball 32 corresponds with a propulsive amount of the linear actuator 42.

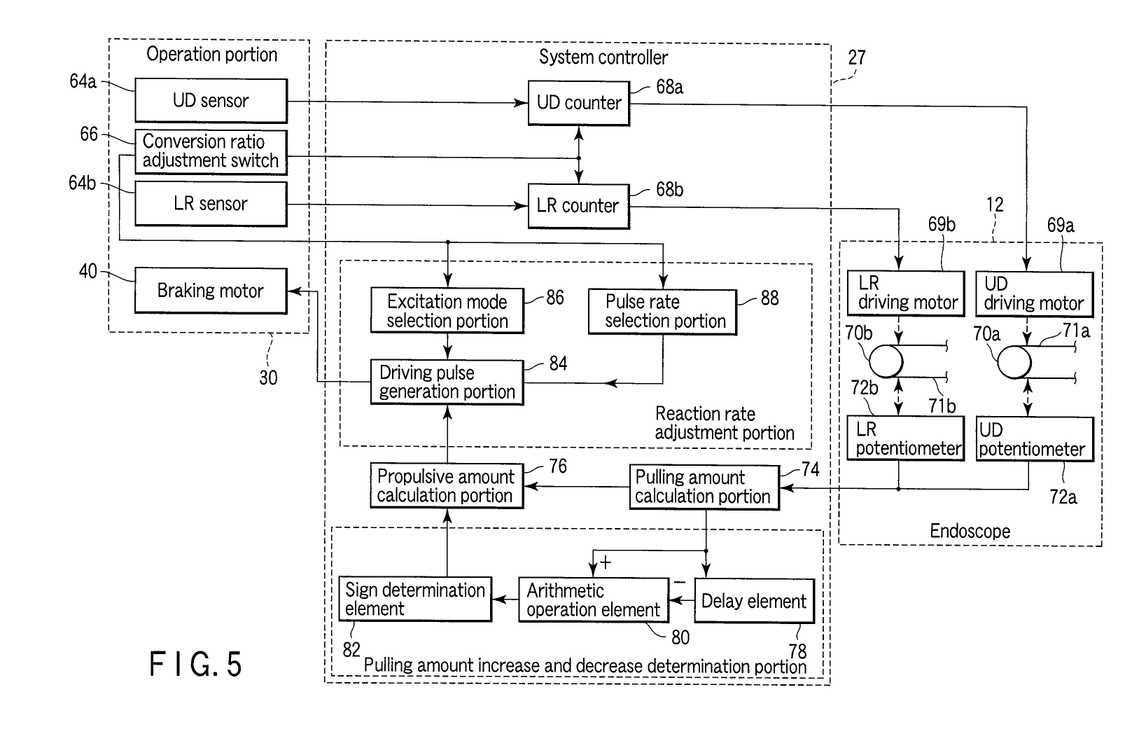

A control system in the endoscope system will now be explained with reference to FIGS. 5 to 8C.

A control system configured to actuate the bending portion 18 to bend in accordance with a rotating operation for the track ball 32 will now be explained with reference to FIG. 5.

A UD sensor 64a and an LR sensor 64b are arranged in the operation portion 30 and are configured to detect an up/down direction component and a left/right direction component in a rotating operation amount to the track ball 32. For example, a non-contact optical sensor is used as each of the UD sensor 64a and the LR sensor 64b. The UD sensor 64a and the LR sensor 64b is configured to generate an up/down direction pulling instruction signal and a left/right direction pulling instruction signal each of which is a pulse signal including a pulse number corresponding with a rotation amount and to output the generated signals to a UD counter 68a and an LR counter 68b of the system controller 27. The UD counter 68a and the LR counter 68b is respectively configured to count pulse numbers of the up/down direction pulling instruction signal and the left/right direction pulling instruction signal, and to increase count values by set counter steps. Moreover, the UD counter 68a and the LR counter 68b is respectively configured to generate an up/down direction pulling order signal and a left/right direction pulling order signal as control order signals each ordering a pulling operation with a pulling amount corresponding with each count value, and to output the generated signals to a UD driving motor 69a and an LR driving motor 69b of the endoscope 12. The UD driving motor 69a and the LR driving motor 69b is respectively configured to rotate a UD sprocket 70a and an LR sprocket 70b in the driving unit 22 in accordance with the up/down direction pulling order signal and the left/right direction pulling order signal to pull and loosen one-end sides and the other end sides of a UD angle wire 71a and an LR angle wire 71b as pulling members wound around the UD sprocket 70a and the LR sprocket 70b, and so the bending portion 18 is actuated to bend in the up/down direction and the left/right direction.

In this manner, the UD driving motor 69a, the LR driving motor 69b, the UD sprocket 70a, and the LR sprocket 70b form a pulling portion, and the UD sensor 64a, the LR sensor 64b, the UD counter 68a, and the LR counter 68b form a pulling order generation portion.

A control system configured to adjust a conversion ratio of a bending amount of the bending portion 18 with respect to a rotating operation amount to the track ball 32 will now be explained with reference to FIG. 5.

The conversion ratio increasing switch 34 and the conversion ratio decreasing switch 36 (which will be generically referred to as a conversion ratio adjustment switch 66 hereinafter) of the operation portion 30 is configured to output a conversion ratio increase and decrease signal to the UD counter 68a and the LR counter 68b in the system controller 27. The UD counter 68a and the LR counter 68b is configured to increase and decrease count steps in accordance with the conversion ratio increase and decrease signal input from the conversion ratio adjustment switch 66. As explained above, the UD counter 68a and the LR counter 68b is respectively configured to count the pulse numbers of the up/down direction pulling instruction signal and the left/right direction pulling instruction signal of the pulse signals including the pulse numbers corresponding with the rotation amount, and to increase the count values by the set counter steps, and to generate the up/down direction pulling order signal and the left/right direction pulling order signal ordering pulling actuations with pulling amounts corresponding with the count values. Therefore, pulling amounts of the UD angle wire 71a and the LR angle wire 71b with respect to a rotating operation amount for the track ball 32, i.e., a conversion ratio of a bending amount of the bending portion 18 is to be increased and decreased in accordance with an increase and decrease in count steps.

In this manner, the UD sensor 64a and the LR sensor 64b form a operation amount detecting portion, the UD counter 68a and the LR counter 68b form a pulling amount setting portion, and the conversion ratio adjustment switch 66 forms a conversion ratio adjustment portion.

A control system configured to control the braking portion in accordance with bending amounts of the bending portion 18 will now be explained with reference to FIGS. 5 to 8C.

Referring to FIG. 5, bending amounts of the bending portion 18 in the up/down direction and the left/right direction corresponds with pulling amounts of the UD angle wire 71a and the LR angle wire 71b, and the pulling amounts of the UD angle wire 71a and the LR angle wire 71b corresponds with rotation amounts of the UD sprocket 70a and the LR sprocket 70b. The rotation amounts of the UD sprocket 70a and the LR sprocket 70b are to be detected by a UD potentiometer 72a and an LR potentiometer 72b, and up/down direction rotation amount data and left/right direction rotation amount data are to output from the UD potentiometer 72a and the LR potentiometer 72b to a pulling amount calculation portion 74. The pulling amount calculation portion 74 is configured to calculate an up/down direction pulling amount P_{UD} and a left/right direction pulling amount P_{LR} from the up/down direction rotation amount data and the left/right direction rotation amount data. Moreover, the pulling amount calculation portion 74 is configured to calculate an absolute value P=(P_{LR}²+P_{UD}²)^{1/2} of a pulling vector having the left/right direction pulling amount P_{LR} and the up/down direction pulling amount P_{UD} as components. The absolute value P of the pulling vector will be referred to as a pulling amount hereinafter.

In this manner, the UD potentiometer 72a, the LR potentiometer 72b, and the pulling amount calculation portion 74 form a pulling amount detecting portion.

It is to be noted that a pulling amount may be calculated from the up/down direction pulling order signal and the left/right direction pulling order signal generated by the UD counter 68a and the LR counter 68b.

The pulling amount calculation portion 74 is configured to output pulling amount data to a pulling amount increase and decrease determination portion. In the pulling amount increase and decrease determination portion, a delay element 78 and an arithmetic operation element 80 are configured to calculate a difference in pulling amount, and a sign determination element 82 is configured to determine a sign of the difference in pulling amount, and so an increase and decrease in pulling amount is to be judged. Further, the pulling amount calculation portion 74 is configured to output the pulling amount data and the pulling amount increase and decrease determination portion is configured to output increase and decrease determination data to a propulsive amount calculation portion 76 as a braking force calculation portion. The propulsive amount calculation portion 76 is configured to calculate a target propulsive amount of the linear actuator 42 of the braking portion in accordance with the pulling amount data and the increase and decrease determination data.

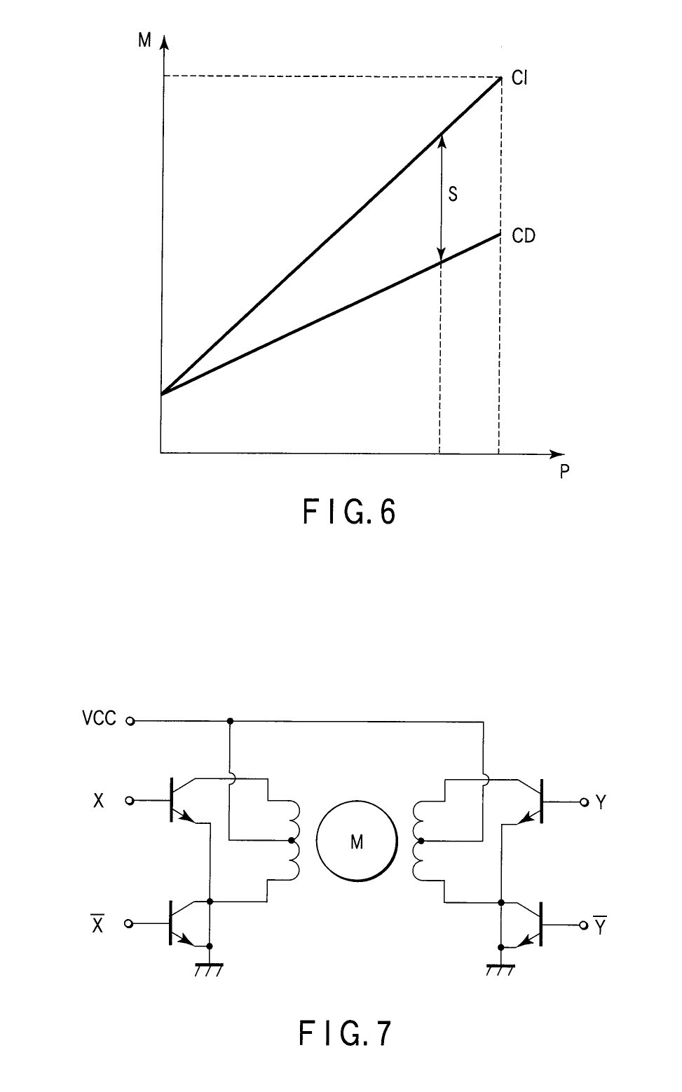

A method of calculating target propulsive amount will now be explained with reference to FIG. 6. The propulsive amount calculation portion 76 stores a propulsive amount characteristic as braking characteristic indicative of a target propulsive amount M with respect to the pulling amount P. As the propulsive amount characteristic, an increase propulsive amount characteristic CI is used when the pulling amount P is determined to be increased, and a decrease propulsive amount characteristic CD is used when the pulling amount P is determined to be decreased. The increase propulsive amount characteristic CI and the decrease propulsive amount characteristic CD are different from each other, and the target propulsive amount M is discontinuously changed when the propulsive amount characteristic is switched between the increase propulsive amount characteristic CI and the decrease propulsive amount characteristic CD. Furthermore, the target propulsive amount M varies in the entire range of a variable region of the pulling amount P. In particular, according to this embodiment, in regard to the increase propulsive amount characteristic CI and the decrease propulsive amount characteristic CD, the target propulsive amount M is a linear function of the pulling amount P, a target propulsive amount M is set in a case where the pulling amount P is zero, and an inclination of the increase propulsive amount characteristic CI is larger than an inclination of the decrease propulsive amount characteristic CD.

Therefore, the target propulsive amount M relatively precipitously rises with an increase in the pulling amount P in accordance with the increase propulsive amount characteristic CI, and it relatively moderately drops with a decrease in the pulling amount P in accordance with the decrease propulsive amount characteristic CD. Furthermore, as indicated by an arrow S in the drawing, the target propulsive amount M is discontinuously reduced when the pulling amount P is changed from an increasing state to a reducing state, and it is discontinuously increased when the pulling amount P is changed from the reducing state to the increasing state.

It is to be noted that a non-linear function such as a quadratic function or a quartic function may be used as the propulsive amount characteristics.

Again referring to FIG. 5, the propulsive amount calculation portion 76 is configured to output target propulsive amount data to a driving pulse generation portion 84 of a reaction rate adjustment portion. On the other hand, the conversion ratio adjustment switch 66 in the operation portion 30 are configured to output a conversion ratio increase and decrease signal to an excitation mode selection portion 86 and a pulse rate selection portion 88 in the reaction rate adjustment portion. The excitation mode selection portion 86 and the pulse rate selection portion 88 is configured to select an excitation mode and a pulse rate in accordance with a conversion ratio and to output excitation mode data and pulse rate data to the driving pulse generation portion 84. The driving pulse generation portion 84 is configured to generate a driving pulse in accordance with the target propulsive amount data, the excitation mode data, and the pulse rate data, and to output the driving pulse to the braking motor 40, and so the braking motor 40 is to be driven.

In this manner, the propulsive amount calculation portion 76 and the driving pulse generation portion 84 form a braking control portion.

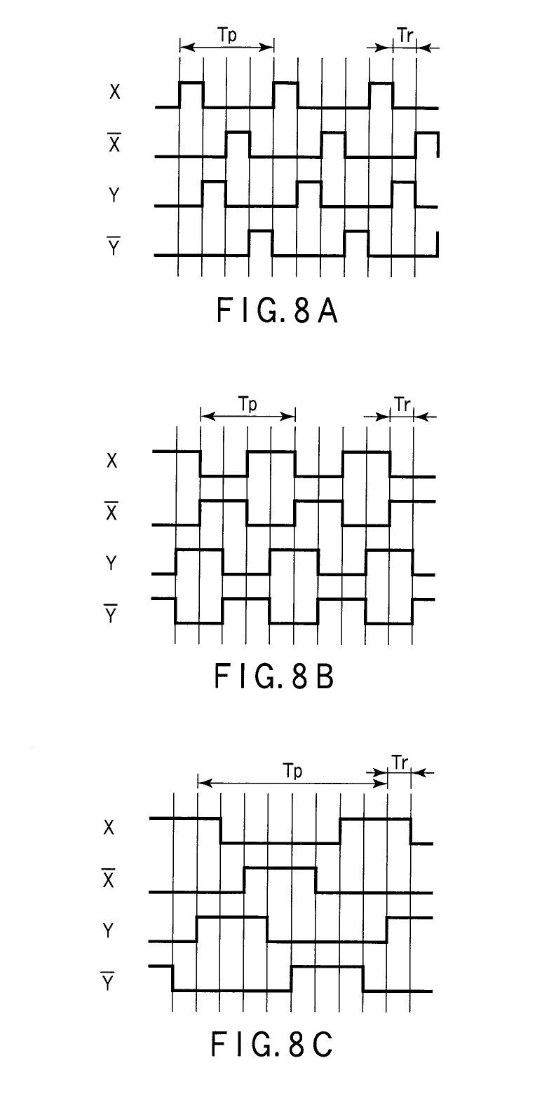

The driving pulse generated by the driving pulse generation portion 84 will now be explained with reference to FIGS. 7 to 8C.

The braking motor 40 is such a two phase five-terminal stepping motor as shown in FIG. 7. As a control mode of the braking motor 40, it is possible to use a single phase excitation mode with a low power consumption shown in FIG. 8A, a two phase excitation mode with a large torque shown in FIG. 8B, and a single-two phase excitation mode with a high accurateness shown in FIG. 8C. In FIGS. 8A to 8C, T_{R} denotes a pulse rate, and T_{P} designates a rotation cycle. That is, four pulses form one rotation cycle in the single phase excitation mode and the two phase excitation mode, and eight pulses form one rotation cycle in the single-two phase excitation mode.

A pulse number of the driving pulse is to be determined based on the target propulsive amount. The excitation mode and the pulse rate are to be selected such that a reaction rate of the braking portion is increased and decreased in accordance with an increase and decrease in conversion ratio. That is, in the case where the braking motor 40 is first driven in the two phase excitation mode with a large torque and then driven in the single-two phase excitation mode with a high accurateness near the target propulsive amount, percentages of numbers of revolutions in the two phase excitation mode and the single-two phase excitation mode with respect to a total number of revolutions of the braking motor 40 are to be changed, and thereby a time required to realize the target propulsive amount is to be changed, and so the reaction rate is to be varied. Specifically, when the number of revolutions in the two phase excitation mode is increased and the number of revolutions in the single-two phase excitation mode is reduced, the time required to realize the target propulsive amount is decreased, and so the reaction rate is increased. On the other hand, when the number of revolutions in the two phase excitation mode is reduced and the number of revolutions in the single-two phase excitation mode is increased, the time required to realize the target propulsive amount is increased, and so the reaction rate is decreased. Additionally, when a pulse rate is increased, the time required to realize the target propulsive amount is increased, and so the reaction rate is reduced. On the other hand, when the pulse rate is reduced, the time required to realize the target propulsive amount is reduced, and so the reaction rate is increased.

A method of using the endoscope system according to this embodiment will now be explained.

The insertion portion 14 of the endoscope 12 is inserted into the interior of the body to observe the interior of the body. The track ball 32 in the operation portion 30 is operated to rotate as required, and so the bending portion 18 is actuated to bend in upper, lower, left, and right directions. Here, the track ball 32 has an infinite operation range, and therefore it is difficult to recognize a bending amount of the bending portion 18 from an operating position of the track ball 32. However, since a braking force to the track ball 32 is increased or decreased and so a resistance force against a rotating operation to the track ball 32 is increased or decreased as the bending amount of the bending portion 18 is increased or reduced, it is possible to recognize the bending amount based on the resistance force. Moreover, when the bending amount of the bending portion 18 is changed from an increasing state to a reducing state or from the reducing state to the increasing state, the braking characteristic of the braking force with respect to the bending amount are switched between the increase breaking characteristic and the decrease braking characteristic different from each other, and the resistance force against the rotating operation to the track ball 32 is discontinuously reduced or increased. Therefore, it is possible to recognize a change of the bending amount from the increasing state to the reducing state or from the increasing state to the reducing state based on a change in the resistance force.

There may be a case where rapidly or finely bending the bending portion 18 is desired in accordance with an operator's preference or whether the bending portion 18 is present near a diseased part. In such a case, the conversion ratio adjustment switch 66 is operated, and a conversion ratio of a bending amount of the bending portion 18 with respect to an operation amount to the track ball 32 is increase or decrease to enable to actuate the bending portion 18 to bend rapidly or finely. At this time, since a reaction rate of the braking portion for generation of a resistance force is increased or reduced with an increase and decrease in the conversion ratio, it is possible to prevent a change in resistance force with respect to a change in bending amount from becoming too late or rapid beyond necessity.

Therefore, the endoscope system according to this embodiment demonstrates the following effect.

In the endoscope system according to this embodiment, an increase and decrease in bending amount of the bending portion 18 is determined, the braking portion configured to apply a braking force to the track ball 32 to generate a resistance force against a rotating operation to the tracking ball 32 is controlled in accordance with a determination result, and it is possible to recognize an increase and decrease direction of the bending amount from a change in resistance force against the rotating operation to the track ball 32. In particular, the braking characteristic of the braking force with respect to the bending amount is switched between the increase braking characteristic and the decrease braking characteristic different from each other in accordance with changeover of the bending amount between an increasing state and a reducing state, and the resistance force against the rotating operation to the track ball 32 is discontinuously changed. Therefore, it is possible to readily recognize that the bending amount is changed over between the increasing state and the reducing state. Additionally, the braking force is changed and the resistance force against the rotating operation to the track ball 32 is changed, in accordance with the bending amount over the entire variable range of the bending amount. Therefore, it is possible to recognize the bending amount from the resistance force against an operation to the track ball 32.

Further, it is possible to adjust the conversion ratio of the bending amount of the bending portion 18 with respect to a rotating operation amount to the track ball 32 to a conversion ratio desired by an operator, and a reaction rate of the braking portion configured to generate the resistance force in accordance with the bending amount is adjusted in response to adjustment of the conversion ratio. Therefore, operability of the endoscope system is improved.

Further, since a propulsive force of the linear actuator 42 is increased by the link mechanism in the braking portion, it is possible to use the small linear actuator with a relatively small output and to reduce an entire size of the braking portion.

Furthermore, since the track ball 32 is dimpled, even when the track ball 32 is operated to rotate with a slippery globed hand and the resistance force against an operation to the track ball 32 becomes relatively high, it is possible to operate the track ball 32 assuredly without slippage.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A pulling apparatus comprising:
an input portion configured to be operated;
a braking portion configured to apply a braking force to the input portion to generate a resistance force against to an operation to the input portion;
a pulling portion configured to pull a pulling member;
a pulling order generation portion configured to generate a control order signal to the pulling portion such that the pulling member is pulled by the pulling portion in accordance with an operation to the input portion;
a pulling amount detecting portion configured to detect pulling amount of the pulling member;
a pulling amount increase and decrease determination portion configured to determine an increase and decrease in the pulling amount detected by the pulling amount detecting portion; and
a braking control portion configured to control the braking portion in accordance with a determination result obtained by the pulling amount increase and decrease determination portion.

2. The pulling apparatus according to claim 1,
wherein the braking control portion includes a braking force calculation portion, and
the braking force calculation portion is configured to calculate a target value of a braking force to be applied to the input portion by the braking portion based on a braking characteristic indicative of a characteristic of the braking force with respect to a pulling amount in accordance with the pulling amount detected by the pulling amount detecting portion, and is configured to switch the braking characteristic between an increase braking characteristic and a decrease braking characteristic different from each other in cases where the pulling amount increase and decrease determination portion determines that the pulling amount is increased and reduced.

3. The pulling apparatus according to claim 2,
wherein the braking characteristic is a braking characteristic so as to change the braking force over an entire variable range of the pulling amount in accordance with the pulling amount.

4. The pulling apparatus according to claim 1,
wherein the pulling order generation portion includes:
an operation amount detecting portion configured to detect an operation amount to the operation portion;
a pulling amount setting portion configured to convert the operation amount detected by the operation amount detecting portion with a conversion ratio to set a target value of the pulling amount of the pulling member by the pulling portion; and
a conversion ratio adjustment portion configured to adjust the conversion ratio.

5. The pulling apparatus according to claim 4,
wherein the braking control portion includes a reaction rate adjustment portion configured to adjust a reaction rate of the braking portion in accordance with the conversion ratio.

6. The pulling apparatus according to claim 1,
wherein the braking portion includes:
a linear actuator configured to be controlled by the braking control portion to generate a propulsive force;
a braking member configured to apply the braking force to the input portion; and
a link mechanism configured to increase and transmit the propulsive force of the linear actuator to the braking member.

7. The pulling apparatus according to claim 1,
wherein the input portion includes a track ball, and
the track ball includes a friction increasing portion formed on an outer surface of the track ball.

8. An endoscope system comprising:
an input portion configured to be operated;
a braking portion configured to apply a braking force to the input portion to generate a resistance force against an operation to the input portion;
a pulling member;
a pulling portion configured to pull the pulling member;
a pulling order generation portion configured to generate a control order signal to the pulling portion such that the pulling member is pulled by the pulling portion in accordance with an operation to the input portion;
a bending portion configured to be actuated to bend by the pulling member in accordance with pulling of the pulling member by the pulling portion;
a pulling amount detecting portion configured to detect pulling amount of the pulling member;
a pulling amount increase and decrease determination portion configured to determine an increase and decrease in the pulling amount detected by the pulling amount detecting portion; and
a braking control portion configured to control the braking portion in accordance with a determination result obtained by the pulling amount increase and decrease determination portion.

9. The endoscope system according to claim 8,
wherein the braking control portion includes a braking force calculation portion, and
the braking force calculation portion is configured to calculate a target value of a braking force to be applied to the input portion by the braking portion based on a braking characteristic indicative of a characteristic of the braking force with respect to a pulling amount in accordance with the pulling amount detected by the pulling amount detecting portion, and is configured to switch the braking characteristic between an increase braking characteristic and a decrease braking characteristic different from each other in cases where the pulling amount increase and decrease determination portion determines that the pulling amount is increased and reduced.

10. The endoscope system according to claim 9,
wherein the braking characteristics is a braking characteristic so as to change the braking force over an entire variable range of the pulling amount in accordance with the pulling amount.

11. The endoscope system according to claim 8,
wherein the pulling order generation portion includes:
an operation amount detecting portion configured to detect an operation amount to the operation portion;
a pulling amount setting portion configured to convert the operation amount detected by the operation amount detecting portion with a conversion ratio to set a target value of the pulling amount of the pulling member by the pulling portion; and
a conversion ratio adjustment portion configured to adjust the conversion ratio.

12. The endoscope system according to claim 11,
wherein the braking control portion includes a reaction rate adjustment portion configured to adjust a reaction rate of the braking portion in accordance with the conversion ratio.

13. The endoscope system according to claim 8,
wherein the braking portion includes:
a linear actuator configured to be controlled by the braking control portion to generate a propulsive force;
a braking member configured to apply the braking force to the input portion; and
a link mechanism configured to increase and transmit the propulsive force of the linear actuator to the braking member.

14. The endoscope system according to claim 8,
wherein the input portion includes a track ball, and
the track ball includes a friction increasing portion formed on an outer surface of the track ball.
